(19) United States
(12) Patent Application Publication
(10) Pub. No.: US 2008/0289624 A1
(43) Pub. Date: Nov. 27, 2008

(54) Aerosol Canister Employing a Polymeric Film Having Improved Moisture Barrier Properties

(76) Inventor: William Christopher Kidd III, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/097,801

(22) PCT Filed: Dec. 18, 2006
(86) PCT No.: PCT/US2006/062218

§ 371 Date: Jun. 17, 2008

Related U.S. Application Data
(60) Provisional application No. 60/752,679, filed on Dec. 21, 2005.

(51) Int. Cl. A61M 15/00

(52) U.S. Cl. 128/200.23

(57) ABSTRACT

A metered dose inhaler for delivering a pharmaceutical aerosol formulation comprises a canister containing the pharmaceutical aerosol formulation therein; a ferrule attached to said canister, the ferrule comprising a valve body having at least one opening therein to allow a quantity of the pharmaceutical aerosol formulation to pass from the container into the valve; and a polymeric film positioned between the ferrule and the canister, the polymeric film being present so as to serve as a moisture barrier relative to said canister.

### FIELD OF THE INVENTION

The present invention generally relates to aerosol canisters used in conjunction with metered dose inhalers for dispensing pharmaceutical aerosol formulations therefrom.

### BACKGROUND OF THE INVENTION

For environmental reasons, there has been a move to replace chlorofluorocarbons (CFCs) (also simply known as “fluorocarbons”) such as P11, P114 and P12 with hydrofluoroalkane propellants such as HFA-134a and HFA-227. When these hydrofluoroalkane propellants are used as a propellant in a pressurized drug delivery system, various technical problems can occur with various drug formulations. Also, it is necessary to modify the construction of metered dose inhalers for optimum stability and aerosol formation.

Non-CFC propellants, especially HFC 134a, are believed to have a greater water solubility than the CFC propellants traditionally used in MDI's. The maximum water solubility in HFC 134a is estimated to be about 2200 ppm whereas for CFC 11, 12 and 114, the maximum water solubilities are about 130 ppm. (See Pischtiak, A. (1999) “Solvay Fluor and Derivate Chemical Data Sheet for CFC and HFC Propellants.”)

This maximum solubility may be further increased if cosolvents such as ethanol are used in the formulation. The mechanism of moisture transport into HFC MDI's has been discussed by Williams, G. and Tcherevatchenkoff, A. (1999) “Moisture Transport Into CFC-Free MDI's,” Respiratory Drug Delivery VI, Hilton Head, S.C., USA.

It is believed that moisture transport is often influenced by the elastomeric nature of the valve gaskets as well as the type of HFA formulation and storage conditions employed. Having the capability to regulate the level of moisture ingress in an inhaler would be highly desirable.

### SUMMARY OF THE INVENTION

The present invention is intended to address the above issue. In one aspect, the invention provides an aerosol inhaler. The inhaler comprises a canister housing a pharmaceutical aerosol formulation therein; a ferrule attached to the canister, the ferrule comprising a valve body having at least one opening therein to allow a quantity of the pharmaceutical aerosol formulation to pass from the container into the valve; and a polymeric film positioned between the ferrule and the canister, the polymeric film being present so as to serve as a barrier to moisture entering the canister.

In another aspect, the invention provides a method for the treatment or prophylaxis of a respiratory disorder. The method comprises administering to a patient by oral inhalation a pharmaceutical aerosol formulation by using the aerosol inhaler.

In another aspect, the invention provides a method of making an aerosol inhaler. The method comprises applying a polymeric film to an outside surface of a canister, attaching a ferrule to the canister, the ferrule comprising a valve body having at least one opening therein, and filling the canister with a pharmaceutical aerosol formulation through the opening of the valve body.

These and other aspects are provided by the present invention.

### BRIEF DESCRIPTION OF THE DRAWINGS

FIG. 1 illustrates a cross-sectional view of a Metered Dose Inhaler in accordance with the present invention.

FIG. 2 illustrates a cross-sectional view of the bottom portion of a Metered Dose Inhaler in accordance with the present invention.

FIG. 3 illustrates Cascade Impaction (CI) fine particle mass data for various inhalers containing polymeric seals.

FIG. 4 illustrates moisture data for various inhalers containing polymeric seals.

### DETAILED DESCRIPTION OF THE INVENTION

Before describing the present invention in detail, it is to be understood that this invention is not limited to particular embodiments described herein. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

It must be noted that, as used in this specification and the appended claims, the singular forms “a,” “an”, “the” and “one” include plural referents unless the content clearly dictates otherwise.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In one aspect, the invention provides an aerosol inhaler. The inhaler comprises a canister housing the pharmaceutical aerosol formulation therein; a ferrule attached to the canister, the ferrule comprising a valve body having at least one opening therein to allow a quantity of the pharmaceutical aerosol formulation to pass from the canister into the valve; and a polymeric film positioned between the ferrule and the canister, the polymeric film being present so as to serve as a barrier to moisture entering the canister.

A number of polymeric films may be employed in accordance with the invention. The polymeric film may be formed from one or more polymers, the selection of which is known to one skilled in the art. For the purposes of the invention, the term “polymeric” should be broadly construed to include, without limitation, homopolymers, copolymers, terpolymers, and the like as well as interpolymers, and blends and combinations of all of the above. Examples of polymers that can be used include, without limitation, thermoplastic polymers. Exemplary polymers that may be employed include, without limitation, polyolefins (e.g., low density polyethylene (LDPE), linear low density polyethylene (LLDPE), medium density polyethylene (MDPE), high density polyethylene (HDPE), ultra high molecular weight polyethylene (UMWPE), and polypropylene (PP)), amorphous and crystalline polyamides, crystalline polyesters, poly(ethylene 2,6-naphthalene dicarboxylate), polycarbonates, methyl methacrylate-styrene copolymer grafted onto a diene elastomer, polyphenylene oxide, polystyrene, polyphenylene oxide/polystyrene blends, poly(vinyl chloride)s, polyacrylates, polymethacrylates, polyalkyl methacrylates, polyethers, polysiloxanes, polysulfones, polyphenylene sulfide, polyether ether ketones, thermoplastic polyimides, polybenzimidazoles, polyquinoxalones, polyoxazolines, styreneacrylonitrile copolymer and acrylonitrile-butadiene-styrene copolymer, vinyl acetate containing polymers, maleic anhydride containing polymers, butadiene and/or isoprene based elastomers, thermotropic liquid crystal polymers based on polyesters, polyamides and polyesteramides, block copolymers of styrenebutadiene and styrene-isoprene, and thermoplastic elastomers. In one embodiment, a polymer containing ethylene-propylene diene monomer (EPDM) can be used. In one embodiment, polyvinylidene chloride resins can be used, e.g., SARAN WRAP® (F-310) made commercially available from S.C. Johnson of Racine, Wis. In one embodiment, a fluorocarbon-based polymer may be used, e.g., polytetrafluoroethylene (PTFE), and in particular a blend of a fluorocarbon polymer and a non-fluorocarbon polymer. An example of such an embodiment is a blend of polytetrafluoroethylene and polyethersulfone (PES) sold commercially as TEFLON® 3200-100 made commercially available from E.I. du Pont de Nemours Company of Wilmington, Del. In one embodiment, the polymeric film may include VAPORCOAT™ 120 made commercially available from Michelman Inc. of Cincinnati Ohio. In one embodiment, the polymeric film may include Valspar Latex (Sealant Lacquer L07505 Grey) made commercially available from The Valspar (Vermicolor) Corporation AG of Gruningen, Switzerland.

The term “film” is to be widely interpreted and refers to a thin sheet of a substance that is in contact with the ferrule and container. The polymeric film may be formed according to techniques known in the art.

The polymeric film may be applied to the canister by employing methods known to the skilled artisan. As an example, the polymeric film may be sprayed to the outer surface of the container and then heated to assist drying the film. Additionally, in other embodiments, the film may be applied by using a syringe or a brush.

In one embodiment, the aerosol inhaler may be a pressurized inhaler, e.g., a Metered Dose Inhaler (MDI). For the purposes of the invention, a number of MDIs can be employed. The pharmaceutical aerosol formulations delivered from such inhalers also are numerous. In various embodiments, the formulations may be employed in or as suspensions or as aerosols delivered from pressurised packs, with the use of a suitable propellant, e.g., a hydrofluoroalkane (HFA) (e.g., 1,1,1,2,3,3,3-heptafluoropropane, 1,1,1,2-tetrafluoroethane), carbon dioxide or other suitable gases.

Exemplary MDIs typically include canisters suitable for delivering the pharmaceutical aerosol formulations. Canisters generally comprise a container capable of withstanding the vapor pressure of the propellant used such as a plastic or plastic-coated glass bottle or preferably a metal can, for example, an aluminum can which may optionally be anodised, lacquer-coated and/or plastic-coated, which container is closed with a metering valve. Aluminum cans which have their inner surfaces coated with a fluorocarbon polymer are particularly preferred. Such polymers can be made of multiples of the following monomeric units: tetrafluoroethylene (PTFE), fluorinated ethylene propylene (FEP), perfluoroalkoxyalkane (PFA), ethylene tetrafluoroethylene (EFTE), vinyldienefluoride (PVDF), and chlorinated ethylene tetrafluoroethylene. Additionally, in other embodiments, cans having inner surfaces coated with blends of fluorocarbon polymers and non-fluorocarbon polymers may also be employed s. Embodiments of coatings used on all or part of the internal surfaces of an MDI are set forth in U.S. Pat. Nos. 6,131,566; 6,143,277; 6,149,892; 6,253,762; 6,511,652; 6,511,653; 6,524,555; 6,532,955; and 6,546,928.

MDIs may also include metering valves designed to deliver a metered amount of the formulation per actuation and incorporate a gasket to prevent leakage of propellant through the valve. The gasket may comprise any suitable elastomeric material such as, for example, low density polyethylene, chlorobutyl, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak plc, UK (e.g. BK300, BK356) and 3M-Neotechnic Ltd, UK (e.g. Spraymiser™). Embodiments of metering valves are set forth in U.S. Pat. Nos. 6,170,717; 6,315,173; and 6,318,603.

In various embodiments, the MDIs may also be used in conjunction with other structures such as, without limitation, overwrap packages for storing and containing the MDIs, including those described in U.S. Pat. Nos. 6,119,853; 6,179,118; 6,315,112; 6,352,152; 6,390,291; 6,679,374, as well as dose counter units such as, but not limited to, those described in U.S. Pat. Nos. 6,360,739 and 6,431,168.

The pharmaceutical aerosol formulation according to the invention includes at least one medicament and at least one propellant, typically an HFA propellant. Medicaments, for the purposes of the invention, include a variety of pharmaceutically active ingredients, such as, for example, those which are useful in inhalation therapy. In general, the term “medicament” is to be broadly construed and include, without limitation, actives, drugs and bioactive agents, as well as biopharmaceuticals. Various embodiments may include medicament present in micronized form. Appropriate medicaments may thus be selected from, for example, analgesics, (e.g., codeine, dihydromorphine, ergotamine, fentanyl or morphine); anginal preparations, (e.g., diltiazem); anti-allergics, (e.g., cromoglicate, ketotifen or nedocromil); antiinfectives (e.g., cephalosporins, penicillins, streptomycin, sulphonamides, tetracyclines and pentamidine); antihistamines, (e.g., methapyrilene); anti-inflammatories, (e.g., anti-inflammatory steroids, beclomethasone (e.g. beclomethasone dipropionate), fluticasone (e.g. fluticasone propionate), flunisolide, budesonide, rofleponide, mometasone (e.g. mometasone furoate), ciclesonide, triamcinolone (e.g. triamcinolon acetonide), 6α,9α-difluoro-11β-hydroxy-16α-methyl-3-oxo-17α-propionyloxy-androsta-1,4-diene-17β-carbothioic acid S-(2-oxo-tetrahydro-furan-3-yl) ester), (6α,11β,16α,17α)-6,9-difluoro-17-{[(fluoromethyl)thio]carbonyl}-1′-hydroxy-16-methyl-3-oxoandrosta-1,4-dien-17-yl 2-furoate, and (6α,11β,16α,17α)-6,9-difluoro-17-{[(fluoromethyl)thio]carbonyl}-11-hydroxy-16-methyl-3-oxoandrosta-1,4-dien-17-yl 4-methyl-1,3-thiazole-5-carboxylate); antitussives, (e.g., noscapine); bronchodilators, (e.g., albuterol (e.g. as sulphate), salbutamol (e.g. as the free base or the sulphate salt), salmeterol (e.g. as xinafoate), ephedrine, adrenaline, fenoterol (e.g as hydrobromide), bitolterol, formoterol (e.g., as fumarate), isoprenaline, metaproterenol, phenylephrine, phenylpropanolamine, pirbuterol (e.g., as acetate), reproterol (e.g., as hydrochloride), rimiterol, terbutaline (e.g., as sulphate), isoetharine, tulobuterol, 4-hydroxy-7-[2-[[2-[[3-(2-(henylethoxy)propyl]sulfonyl]ethyl]-amino]ethyl-2(3H)-benzothiazolone), 3-(4-{[6-({(2R)-2-hydroxy-2-[4-hydroxy-3-(hydroxymethyl) phenyl]ethyl}amino)hexyl]oxy}butyl) benzenesulfonamide, 3-(3-{[7-({(2R)-2-hydroxy-2-[4-hydroxy-3-(hydroxymethyl)phenyl]ethyl}amino)heptyl]oxy}propyl) benzenesulfonamide, 4-{(1R)-2-[(6-{2-[(2,6-dichlorobenzyl)oxy]ethoxy}hexyl)amino]-1-hydroxyethyl}-2-(hydroxymethyl)phenol, 2-hydroxy-5-((1R)-1-hydroxy-2-{[2-(4-{[(2R)-2-hydroxy-2-phenylethyl]amino}phenyl)ethyl]amino}ethyl)phenylformamide, and 8-hydroxy-5-{(1R)-1-hydroxy-2-[(2-{4-[(6-methoxy-1,1′-biphenyl-3-yl)amino]phenyl}ethyl)amino]ethyl}quinolin-2(1H)-one); diuretics, (e.g., amiloride); anticholinergics, (e.g., ipatropium (e.g., as bromide), tiotropium, atropine or oxitropium); hormones, (e.g., cortisone, hydrocortisone or prednisolone); xanthines, (e.g., aminophylline, choline theophyllinate, lysine theophyllinate or theophylline); therapeutic proteins and peptides, (e.g., insulin). In addition to those stated above, it will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimize the activity and/or stability of the medicament. It will be further clear to a person skilled in the art that where appropriate, the medicaments may be used in the form of a pure isomer, for example, R-salbutamol or RR-formoterol.

Particular medicaments for administration using pharmaceutical formulations in accordance with the invention include anti-allergics, bronchodilators, beta agonists (e.g., long-acting beta agonists), and anti-inflammatory steroids of use in the treatment of respiratory conditions, as defined herein, by inhalation therapy, for example, cromoglicate (e.g. as the sodium salt), salbutamol (e.g. as the free base or the sulphate salt), salmeterol (e.g. as the xinafoate salt), bitolterol, formoterol (e.g. as the fumarate salt), terbutaline (e.g. as the sulphate salt), 3-(4-{[6-({(2R)-2-hydroxy-2-[4-hydroxy-3-(hydroxymethyl)phenyl]ethyl}amino)hexyl]oxy}butyl)benzenesulfonamide,
3-(3-{[7-({(2R)-2-hydroxy-2-[4-hydroxy-3-(hydroxymethyl)phenyl]ethyl}amino) heptyl]oxy}propyl)benzenesulfonamide, 4-{(1R)-2-[(6-{2-[(2,6-dichlorobenzyl)oxy]ethoxy}hexyl)amino]-1-hydroxyethyl}2-(hydroxymethyl)phenol, 2-hydroxy-5-((1R)-1-hydroxy-2-{[2-(4-{[(2R)-2-hydroxy-2-phenylethyl]amino}phenyl)ethyl]amino}ethyl)phenylformamide, 8-hydroxy-5-{(1R)-1-hydroxy-2-[(2-{4-[(6-methoxy-1,1′-biphenyl-3-yl)amino]phenyl}ethyl)amino]ethyl}quinolin-2(1H)-one, reproterol (e.g. as the hydrochloride salt), a beclomethasone ester (e.g. the dipropionate), a fluticasone ester (e.g. the propionate), a mometasone ester (e.g., the furoate), budesonide, dexamethasone, flunisolide, triamcinolone, tripredane, (22R)-6α,9α-difluoro-11β,21-dihydroxy-16α,17α-propylmethylenedioxy-4-pregnen-3,20-dione. Medicaments useful in erectile dysfunction treatment (e.g., PDE-V inhibitors such as vardenafil hydrochloride, along with alprostadil and sildenafil citrate) may also be employed. It should be understood that the medicaments that may be used in conjunction with the inhaler are not limited to those described herein.

Salmeterol, especially salmeterol xinafoate, salbutamol, fluticasone propionate, beclomethasone dipropionate and physiologically acceptable salts and solvates thereof are especially preferred.

It will be appreciated by those skilled in the art that the formulations according to the invention may, if desired, contain a combination of two or more of any of the above medicaments. As an example, formulations containing two active ingredients are known for the treatment and/or prophylaxis of respiratory disorders such as those described herein, for example, formoterol (e.g. as the fumarate) and budesonide, salmeterol (e.g. as the xinafoate salt) and fluticasone (e.g. as the propionate ester), salbutamol (e.g. as free base or sulphate salt) and beclomethasone (as the dipropionate ester) are preferred.

In one embodiment, a particular combination that may be employed is a combination of a beta agonist (e.g., a long-acting beta agonist) and an anti-inflammatory steroid. One embodiment encompasses a combination of salmeterol, or a salt thereof (particularly the xinafoate salt) and fluticasone propionate. The ratio of salmeterol to fluticasone propionate in the formulations according to the present invention is preferably within the range 4:1 to 1:20. The two drugs may be administered in various manners, simultaneously, sequentially, or separately, in the same or different ratios. In various embodiments, each metered dose or actuation of the inhaler will typically contain from 25 μg to 100 μg of salmeterol and from 25 μg to 500 μg of fluticasone propionate.

The pharmaceutical formulation may be administered according to various occurrences per day. In one embodiment, the pharmaceutical formulation may be administered twice daily. In one embodiment, the pharmaceutical formulation may be administered once daily.

Embodiments of specific medicament combinations that may be used in various pharmaceutical formulations are as follows:

1) fluticasone propionate 100 μg/salmeterol 50 μg

2) fluticasone propionate 250 μg/salmeterol 50 μg

3) fluticasone propionate 500 μg/salmeterol 50 μg

In another aspect, the present invention provides a method for the prophylaxis or treatment of a respiratory disorder in a patient. As an example, the present invention provides such a method for the prophylaxis or treatment of disorders associated with reversible airways obstruction such as asthma, chronic obstructive pulmonary disease (COPD), respiratory tract infection or upper respiratory tract disease, and rhinitis (e.g., allergic and non-allergic). The method comprises administering an effective amount of a pharmaceutical aerosol formulation from a metered dose inhaler described herein.

The invention will now be described in reference to the embodiments set forth in the drawings. It should be understood that these embodiments serve to illustrate the invention, and do not limit the scope of the invention as described by the claims.

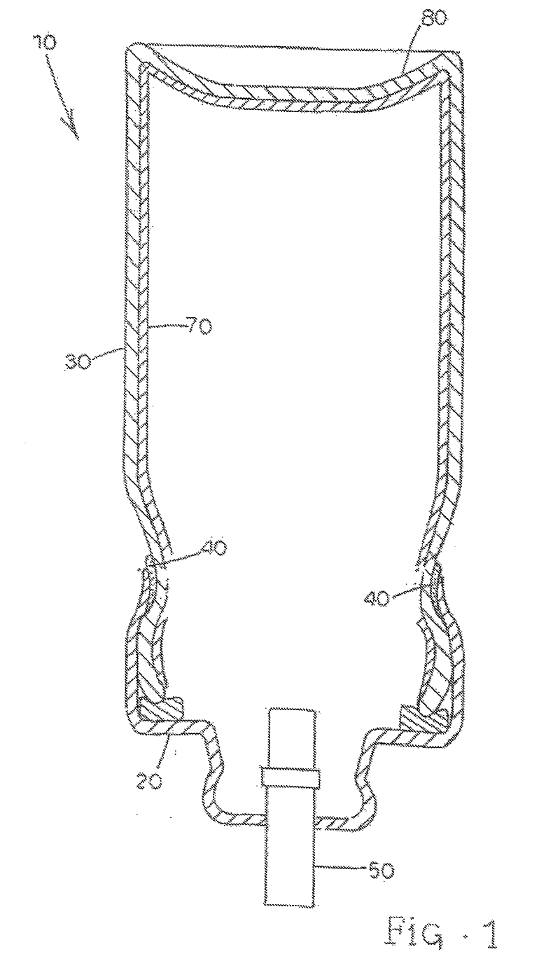

FIG. 1 illustrates a cross-sectional view of a portion of an aerosol inhaler 10 in accordance with the present invention. The inhaler 10 includes a ferrule 20 attached (in this specific embodiment crimped) to a canister 30. As shown, polymeric film 40 is present between ferrule 20 and canister 30 intended to serve as a moisture barrier, and extends throughout the circumference of the canister 30 where indicated. The canister 30 (alternatively referred to as a can or container) may be selected from those that are conventionally used in metered dose inhaler applications. In particular, the canister 30 may be fabricated from a number of materials. Examples of such materials include, without limitation, aluminium, an alloy of aluminium, stainless steel, tin plate, an alloy of copper, glass, or plastic, as well as combinations of the above. Embodiments of containers are described in U.S. Pat. No. 6,253,762.

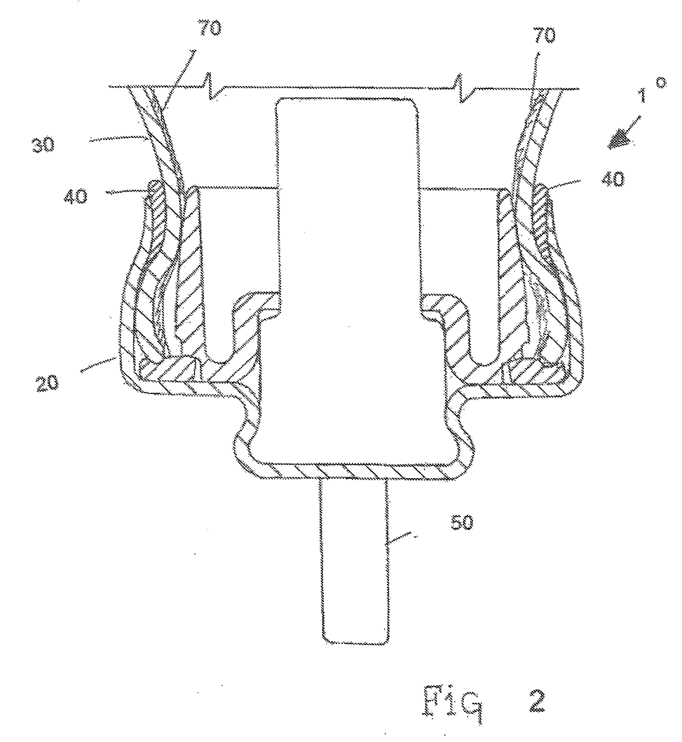

Ferrule 20 may contain a valve body 50 suitable for delivering a pharmaceutical aerosol formulation to a patient. In particular, the valve body may be structured to deliver a metered quantity of pharmaceutical aerosol formulation to the patient. Examples of valve bodies are set forth in U.S. Pat. Nos. 6,170,717; 6,315,173; and 6,318,603. industry, for example, from Valois, France (e.g. DF10, DF30, DF60), Bespak plc, UK (e.g. BK300, BK356) and 3M-Neotechnic Ltd, UK (e.g. Spraymiser™). Additionally, in this embodiment, the canister 30 contains a polymer coating 70 on the inside walls of the canister. Examples of such coatings are set forth in U.S. Pat. Nos. 6,131,566; 6,143,277; 6,149,892; 6,253,762; 6,511,652; 6,511,653; 6,524,555; 6,532,955; and 6,546,928. Additionally, the canister 30 includes base 80 that is shaped substantially ellipsoidal. FIG. 2 illustrates a cross-sectional view of the bottom portion of the inhaler.

The present invention is highly advantageous. By judicious use of a polymeric film, the invention is capable of allowing an aerosol inhaler to exhibit improved moisture and CI performance. Moreover, the polymeric seal is used in a fashion such that it does not come into direct contact with the aerosol formulation. Accordingly, such a feature may be clearly distinguished from a conventional gasket used in an MDI which often contacts the formulation.

The invention will now be described with respect to the following examples. It should be understood that these examples are set forth for the purpose of illustrating the invention and do not limit the invention as defined by the claims. In the examples, the following designations refer to corresponding cans:

“A”: Sprayed Vaporcoat 120—Assembled can

“B”: Sprayed Valspar Latex—Assembled can

“C”: Sprayed Valspar Latex—Open can

“D”: Sprayed Vaporcoat 120—Open can

“E”: Syringe Valspar Latex—Assembled can

“F”: Syringe—Liquid EPDM—Assembled can

“G”: Syringe Valspar Latex—Open can

“H”: Syringe—Liquid EPDM—Open can

“I”: Brush—Saran F-310 Assembled can

“J”: Heat gun—ATUM heat shrinking tube—Assembled can

“K”: Control samples—Assembled can

In reference to the above, the term “assembled can” refers to the polymeric material which forms the film being applied to the canister after the valve was crimped to the canister to attempt to block the gap between the valve and canister. The term “open can” refers to polymeric material which forms the film being applied to the canister prior to crimping the valve. Control samples refer to commercially available canisters employed in Ventolin® HFA made commercially available by GlaxoSmithkline. Polymer spraying was carried out by Sprimag in Germany. Polymer application by syringe was carried out by Sprimag in Germany. Polymer application by brush was carried out by GlaxoSmithkline in Research Triangle Park, N.C. Polymer application via heat shrinking tube was carried out by GlaxoSmithkline in Research Triangle Park, N.C.

### EXAMPLE 1

### Cascade Impaction Results

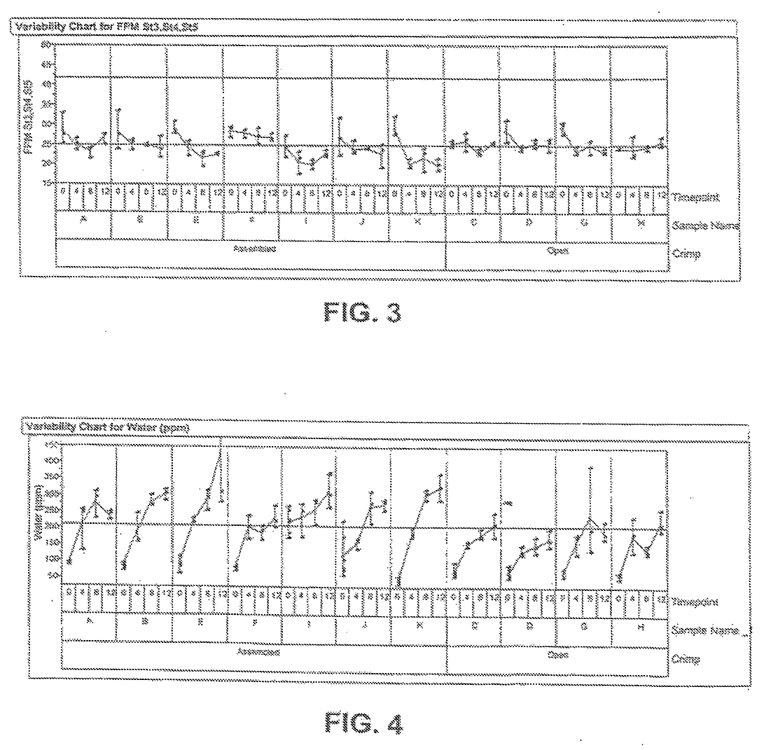

Samples A, B, E, F, I, J, K, C, D, G and H were manufactured and stored for 14 days under dry conditions. These samples were then tested for Cascade Impaction performance using standard methodology. The samples were measured at 0, 4 weeks, 8 weeks and 12 weeks. The results are set forth in FIG. 3. As shown, samples F and H exhibited very good performance.

### EXAMPLE 2

### Moisture Results

The above manufactured and stored samples were tested for moisture using standard methodology. The samples were measured at 0, 4 weeks, 8 weeks and 12 weeks. The results are set forth in FIG. 4.

The invention has been described by the examples and embodiments set forth herein. It should be appreciated that the examples and embodiments are set forth for illustrative purposes, and do not limit the scope of the invention as defined by the claims.

What is claimed is:

1. A metered dose inhaler for delivering a pharmaceutical aerosol formulation, said inhaler comprising:
a canister containing the pharmaceutical aerosol formulation therein;
a ferrule attached to said canister, the ferrule comprising a valve body having at least one opening therein to allow a quantity of the pharmaceutical aerosol formulation to pass from the container into the valve; and
a polymeric film positioned between said ferrule and said canister, the polymeric film being present so as to serve as a moisture barrier relative to said canister.

2. The inhaler according to claim 1, wherein the polymeric film comprises a thermoplastic polymer.

3. The inhaler according to claim 1, wherein the polymeric film comprises EPDM.

4. The inhaler according to claim 1, wherein the polymeric film comprises a vinylidene chloride polymer latex.

5. The inhaler according to claim 1, wherein the pharmaceutical aerosol formulation comprises a hydrofluoroalkane (HFA) propellant and at least one medicament.

6. The inhaler according to claim 5, wherein the propellant is selected from the group consisting of 1,1,1,2-tetrafluoroethane and 1,1,1,2,3,3,3-n-heptafluoropropane, or a mixture thereof.

7. The inhaler according to claim 6, wherein the propellant is 1,1,1,2-tetrafluoroethane.

8. A method for the treatment or prophylaxis of a respiratory disorder, comprising:
administering to a patient by oral inhalation a pharmaceutical aerosol formulation by using the aerosol inhaler according to claim 1.

9. A method of making a metered dose inhaler for dispensing a pharmaceutical aerosol formulation, said method comprising:
applying a polymeric film to an outside surface of a container;
attaching a ferrule to the canister, the ferrule comprising a valve body having at least one opening therein; and
filling the canister with a pharmaceutical aerosol formulation through the opening of the valve body.

10. The method according to claim 9, wherein the polymeric film comprises a thermoplastic polymer.

11. The method according to claim 9, wherein the polymeric film comprises EPDM.

12. The method according to claim 9, wherein the polymeric film comprises a vinylidene chloride polymer latex.
